(19) United States
(12) Patent Application Publication
(10) Pub. No.: US 2004/0073216 A1
(43) Pub. Date: Apr. 15, 2004

(54) Apparatus and method for attaching adjacent bones

(75) Inventor: Isador H. Lieberman, Pepper Pike, OH (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 10/621,015

(22) Filed: Jul. 16, 2003

Related U.S. Application Data
(63) Continuation-in-part of application No. 10/395,779, filed on Mar. 24, 2003.
Application No. 10/395,779 is a continuation-in-part of application No. 09/708,940, filed on Nov. 8, 2000, now Pat. No. 6,551,322.
(60) Provisional application No. 60/238,271, filed on Oct. 5, 2000.

(51) Int. Cl. A61B 17/58

(52) U.S. Cl. 606/61

(57) ABSTRACT

An apparatus for attaching a first bone to an adjacent second bone includes an anchor having a platform and at least two helical spikes for embedding into at least one of the bones upon rotation of the platform. The helical spikes project from a surface on the platforms. The anchor has a first condition in which a first portion of each of the helical spikes is extendable into one of the bones. The anchor further has a second condition in which the first portions are extendable into the other of the bones and a second portion of each of the helical spikes is extendable into the one bone to attach the bones to each other while maintaining a space between the bones. Each of the helical spikes further includes a third portion that extends between the first and second portions and that, when the anchor is embedded into the bones, extends across the space.

### RELATED APPLICATIONS

This application is a continuation-in-part of co-pending U.S. patent application Ser. No. 10/395,779, entitled “APPARATUS FOR IMPLANTATION INTO BONE”, filed Mar. 24, 2003, which is itself a continuation-in-part of U.S. patent application Ser. No. 09/708,940, filed Nov. 8, 2000 and now issued as U.S. Pat. No. 6,551,322, which is based on U.S. Provisional Patent Application Serial No. 60/238,271, filed Oct. 5, 2000.

### FIELD OF THE INVENTION

The present invention an apparatus and method for attaching adjacent bones, and, in particular, is directed to a method and apparatus for attaching a fifth lumbar vertebrae to a sacrum.

### BACKGROUND OF THE INVENTION

Bone screws are used in the medical field for a variety of purposes. Typical uses for bone screws, also referred as anchors, include treating a bone fracture, attaching a corrective device across a group of bones such as vertebrae, and attaching soft tissue, such as a ligament or tendon, to bone.

Most known bone screws use a conventional screw design, i.e. a solid shank, with one or more external thread convolutions. The solid shank and external threads of the conventional bone screws can cause the bone screws to displace and/or destroy an undesirably large amount of bone when implanted. Typically, implantation of a bone screw into bone involves drilling a hole, tapping the hole, and then inserting the screw. In many cases, such drilling and tapping can further damage the bone. Conventional bone screws can also require a large amount of torque to implant the screw into a bone. Further, the low resistance of the conventional screw to being pulled axially from the bone is a function of the relatively small surface area of the bone that interfaces with the screw threads.

It is also known to use a corkscrew-style helical spike as a bone screw or tissue anchor. The known corkscrew-style tissue anchors, when implanted, displace less bone than the conventional bone screws, but are generally not able to withstand high tensile loads without structural failure. European Patent No. 0 374 088 A1 discloses a bone screw having a twin-corkscrew design. In this twin-corkscrew design, which is formed by drilling a passage up through a screw having a solid shank and then machining out the material between the two corkscrews, the junction of the corkscrews with the shank is not capable of structurally withstanding high tensile loads and repetitive fatigue loads.

Many of the known bone screws, such as those described above, can be susceptible to toggling in the bone and can also pull out of the bone due to the substantial forces on the screws from human body movement and muscle memory. In order to achieve a high pull-out resistance, it is common to use additional screws, which results in an undesirably large amount of bone being displaced. In order to achieve a high pull-out resistance, it is also known to thread a bone screw all of the way through a bone and place a nut on the opposite side. However, use of such a nut increases the complexity of the surgical procedure as an additional incision on the opposite side of the body can be required.

One challenge in the field of spine surgery is how to deal with disease or trauma in the area of the fifth lumber (L5) vertebrae and the sacrum. The intervertebral disc that normally separates the L5 vertebrae and the first sacral (S1) vertebrae is prone to problems, such as degeneration and rupture, and various techniques have been developed for treating these problems as well as problems with vertebral collapse in this area. While the known techniques and surgical instrumentation are effective in some cases, improved techniques and instrumentation are needed to deal with other cases. In particular, minimally invasive methods and associated surgical instrumentation for treating L5/S1 disorders are desirable.

### SUMMARY OF THE INVENTION

The present invention is an apparatus for attaching a first bone to an adjacent second bone. The second bone is separated from the first bone by a space between the bone. The apparatus comprises an anchor having a platform for drivingly rotating the anchor and at least two helical spikes for embedding into at least one of the first and second bones upon rotation of the platform. The platform has a first surface that extends generally transverse to a longitudinal axis of the anchor. The at least two helical spikes project from the first surface of the platform and extend around the longitudinal axis. The at least two helical spikes have a tip portion at a distal end which penetrates into bone as the platform is rotated. The anchor has a first condition in which a first portion of each of the at least two helical spikes is extendable into one of the first and second bones. The anchor further has a second condition in which the first portions are extendable into the other of the first and second bones and a second portion of each of the at least two helical spikes is extendable into the one bone to attach the first and second bones to one another while maintaining the space between the bones. Each of the at least two helical spikes further includes a third portion that extends between the first and second portions and that, when the anchor is embedded into the first and second bones, extends across the space between the bones.

The present invention further comprises an apparatus for attaching a fifth lumbar (L5) vertebrae to a sacrum. The apparatus comprises an anchor for extending between the L5 vertebrae and the sacrum and for attaching the L5 vertebrae to the sacrum. The anchor has a platform for drivingly rotating the anchor. The platform includes a first surface that is solid and that extends generally transverse to a longitudinal axis of the anchor. The anchor further has at least two helical spikes for embedding into both of the L5 vertebrae and the sacrum upon rotation of the platform. The at least two helical spikes project from the first surface of the platform and extend around the longitudinal axis. The at least two helical spikes have a tip portion at a distal end for penetrating into at least one of the L5 vertebrae and the sacrum as the platform is rotated. The anchor has a first condition in which the at least two helical spikes are embeddable into one of the L5 vertebrae and the sacrum. The anchor further has a second condition in which the at least two helical spikes are embeddable into both of the L5 vertebrae and the sacrum to attach the L5 vertebrae and the sacrum to one another while maintaining an intervertebral space between the L5 vertebrae and the sacrum. The anchor is movable from the first condition to the second condition by rotation of the platform. A portion of each of the at least two helical spikes of the anchor, when the anchor is embedded into the L5 vertebrae and the sacrum, extends across the intervertebral space between the L5 vertebrae and the sacrum.

In accordance with another aspect of the present invention, a method for attaching a first bone in a patient's body to an adjacent second bone is provided. The second bone is separated from the first bone by a space between the bones. According to the inventive method, an anchor having a platform and at least two helical spikes is provided. The platform has a first surface that extends generally transverse to a longitudinal axis of the anchor, and the at least two helical spikes project from the first surface of the platform and extend around the longitudinal axis. One of the bones is engaged by the at least two helical spikes. The platform of the anchor is then rotated, which embeds a first portion of each of the at least two helical spikes into one of the first and second bones. The platform of the anchor is further rotated so that the anchor extends across the space and embeds the first portion of the anchor into the other of the first and second bones and a second portion of the at least two helical spikes to attach the first and second bones to one another while maintaining the space between the bones, with a portion of each of the at least two helical spikes extending across the space between the bones.

The present invention further provides a method for attaching a fifth lumbar (L5) vertebrae to a sacrum. According to the inventive method, disc material disposed between the L5 vertebrae and the sacrum is removed to create an interbody space. An anchor is provided for extending between the L5 vertebrae and the sacrum and for attaching the L5 vertebrae to the sacrum. The anchor has a platform for drivingly rotating the anchor. The platform includes a first surface that extends generally transverse to a longitudinal axis of the anchor. The anchor further has at least two helical spikes for embedding into both of the L5 vertebrae and the sacrum upon rotation of the platform. The at least two helical spikes project from the first surface and extend around the longitudinal axis. One of the L5 vertebrae and the sacrum is engaged by the at least two helical spikes on the anchor. The platform is then rotated so that a portion of each of the at least two helical spikes embeds into one of the sacrum and the L5 vertebrae. The platform is further rotated so that the at least two helical spikes extend across the interbody space and into the other of the sacrum and the L5 vertebrae to attach the L5 vertebrae and the sacrum to each other while maintaining the interbody space between the L5 vertebrae and the sacrum such that a portion of each of the at least two helical spikes extends across the interbody space between the L5 vertebrae.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic side view of an apparatus for attaching adjacent bones in accordance with a first embodiment of the present invention, the apparatus being shown partially implanted in the first sacral vertebrae;

FIG. 2 is a view similar to FIG. 1 showing the apparatus following implantation;

FIG. 2A is a view of a portion of FIG. 2 and illustrating an alternate position for the apparatus following implantation;

FIG. 3 is an anterior view taken along line 3-3 in FIG. 2;

FIG. 4 is a view similar to FIG. 3 illustrating the implantation of two of the apparatuses;

FIG. 5 is a side view, partly in section, of the apparatus of FIG. 1;

FIG. 6 is a view taken along line 6-6 in FIG. 5;

FIG. 7 is a view similar to FIG. 6 illustrating an alternate construction;

FIG. 8 is a sectional view taken along line 8-8 in FIG. 5;

FIG. 8A is a sectional view similar to FIG. 8 illustrating an alternate construction;

FIG. 9 is a sectional view taken along line 9-9 in FIG. 5;

FIG. 9A is a sectional view similar to FIG. 9 illustrating an alternate construction;

FIG. 10 illustrates an alternate configuration for an end portion of the apparatus of FIG. 1;

FIG. 11 a side view similar to FIG. 5 illustrating an apparatus for attaching adjacent bones in accordance with a second embodiment of the present invention;

FIG. 12 is a view taken along line 12-12 in FIG. 11;

FIG. 13 is a sectional view taken along line 13-13 in FIG. 11;

FIG. 13A is a sectional view similar to FIG. 13 illustrating an alternate construction;

FIG. 14 is a sectional view taken along line 14-14 in FIG. 11;

FIG. 14A is a sectional view similar to FIG. 14 illustrating an alternate construction;

FIG. 15 is a sectional view taken along line 15-15 in FIG. 11;

FIG. 15A is a sectional view similar to FIG. 15 illustrating an alternate construction;

FIG. 16 is a side view similar to FIG. 5 illustrating an apparatus for attaching adjacent bones in accordance with a third embodiment of the present invention;

FIG. 17 is a schematic view showing the apparatus of FIG. 16 following implantation;

FIG. 18 is a side view similar to FIG. 5 illustrating an apparatus for attaching adjacent bones in accordance with a fourth embodiment of the present invention;

FIG. 19 is a schematic view showing the apparatus of FIG. 18 following implantation;

FIG. 20 is a schematic side view of an apparatus for attaching adjacent bones in accordance with a fifth embodiment of the present invention, the apparatus being shown partially implanted in the first sacral vertebrae;

FIG. 21 is a view similar to FIG. 20 showing the apparatus following implantation;

FIG. 22 is a side view of the apparatus of FIG. 20;

FIG. 23 is a view taken along line 23-23 in FIG. 22;

FIG. 24 is a sectional view taken along line 24-24 in FIG. 23;

FIG. 24A is a sectional view similar to FIG. 24 illustrating an alternate construction;

FIG. 25 is a sectional view taken along line 25-25 in FIG. 24;

FIG. 25A is a sectional view similar to FIG. 25 illustrating an alternate construction;

FIG. 26 is a side view, partially in section, illustrating the apparatus of FIG. 22 in a first condition prior to implantation;

FIG. 27 is a view similar to FIG. 26 during implantation of the apparatus;

FIG. 28 is a view similar to FIG. 26 following implantation;

FIG. 29 is a schematic side view of an apparatus for attaching adjacent bones in accordance with a sixth embodiment of the present invention, the apparatus being shown following implantation;

FIG. 30 is an anterior view taken along line 30-30 in FIG. 29;

FIG. 31 is a view similar to FIG. 30 showing the implantation of two of the apparatuses;

FIG. 32 is a schematic side view of an apparatus for attaching adjacent bones in accordance with a seventh embodiment of the present invention, the apparatus being shown following implantation;

FIG. 33 is a schematic side view of an apparatus for attaching adjacent bones in accordance with an eighth embodiment of the present invention, the apparatus being shown following implantation;

FIG. 34 is a schematic side view of an apparatus for attaching adjacent bones in accordance with a ninth embodiment of the present invention, the apparatus being shown following implantation;

FIG. 35 is a schematic side view of an apparatus for attaching adjacent bones in accordance with a tenth embodiment of the present invention, the apparatus being shown following implantation;

FIG. 36 is a posterior view taken along line 36-36 in FIG. 35;

FIG. 37 is a view similar to FIG. 36 showing the implantation of two of the apparatuses;

FIG. 38 is a schematic side view of an apparatus for attaching adjacent bones in accordance with an eleventh embodiment of the present invention, the apparatus being shown following implantation;

FIG. 39 is a schematic side view of an apparatus for attaching adjacent bones in accordance with a twelfth embodiment of the present invention, the apparatus being shown following implantation;

FIG. 40 is a schematic side view of an apparatus for attaching adjacent bones in accordance with a thirteenth embodiment of the present invention, the apparatus being shown following implantation;

FIG. 41 is a side view, partly in section, of a tool for use with the apparatuses of the present invention; and

FIG. 42 illustrates the use of the tool of FIG. 41.

### DESCRIPTION OF EMBODIMENTS

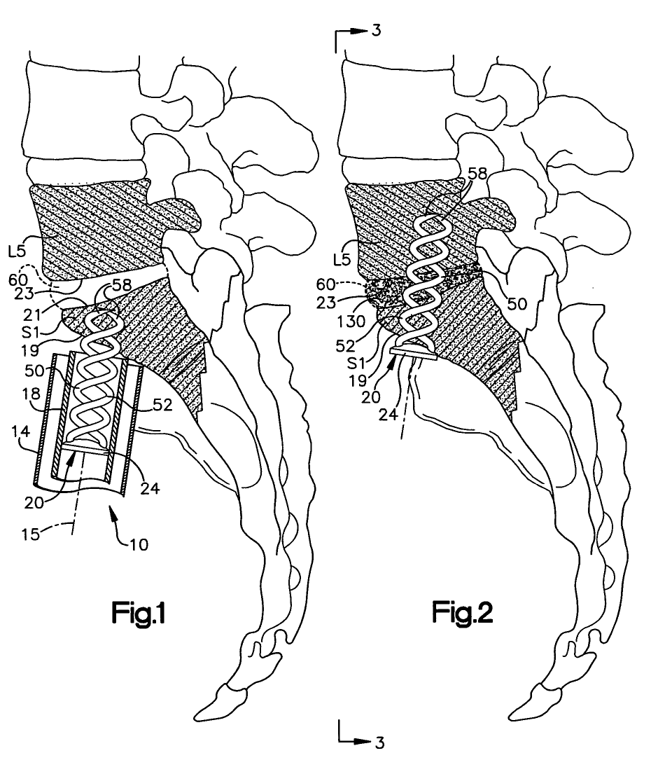

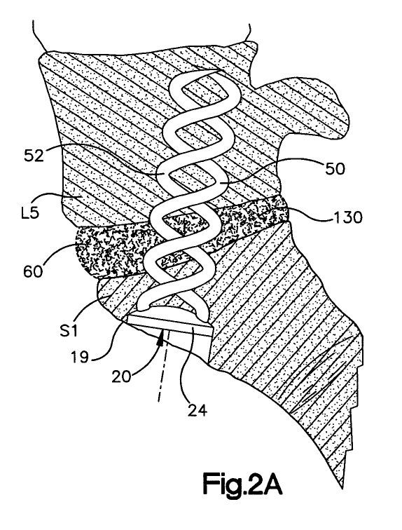

The present invention an apparatus and method for attaching adjacent bones, and, in particular, is directed to a method and apparatus for attaching a fifth lumbar (L5) vertebrae to a sacrum. As representative of the present invention, FIGS. 1 and 2 illustrate an apparatus 10 for attaching adjacent bones in accordance with a first embodiment. In FIG. 1, the apparatus 10 is shown partially implanted in the first sacral (S1) vertebrae, while FIG. 2 illustrates the apparatus 10 fully implanted in the S1 vertebrae and the L5 vertebrae.

The apparatus 10 comprises an anchor 20 made from a biocompatible material, such as titanium or stainless steel. It is contemplated that the biocompatible material used for the anchor 20 could be polymeric or composite (i.e., carbon fiber or other biologic composite) in nature. It is further contemplated that the biocompatible material used to make the anchor 20 could also be biodegradable.

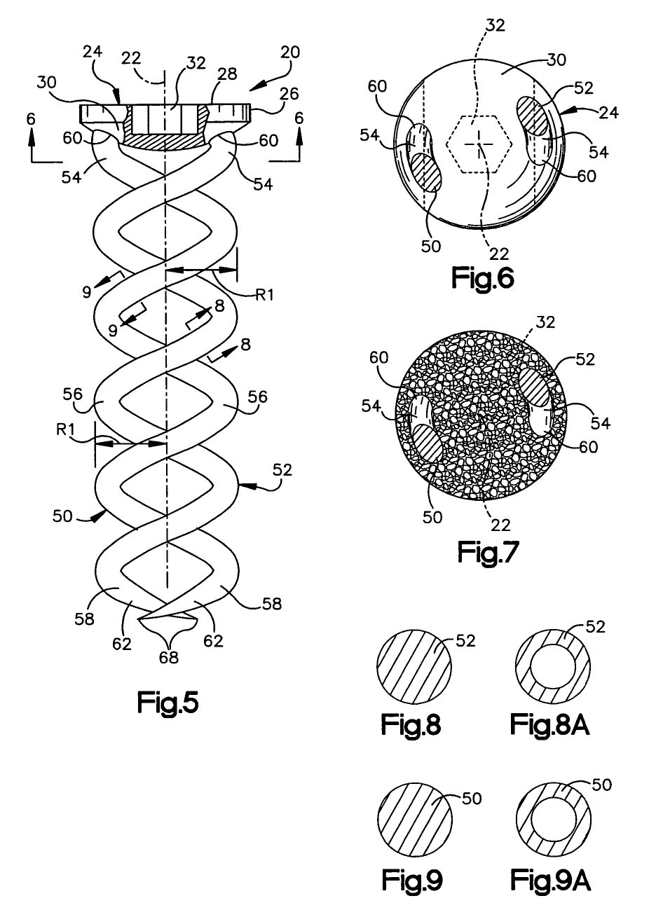

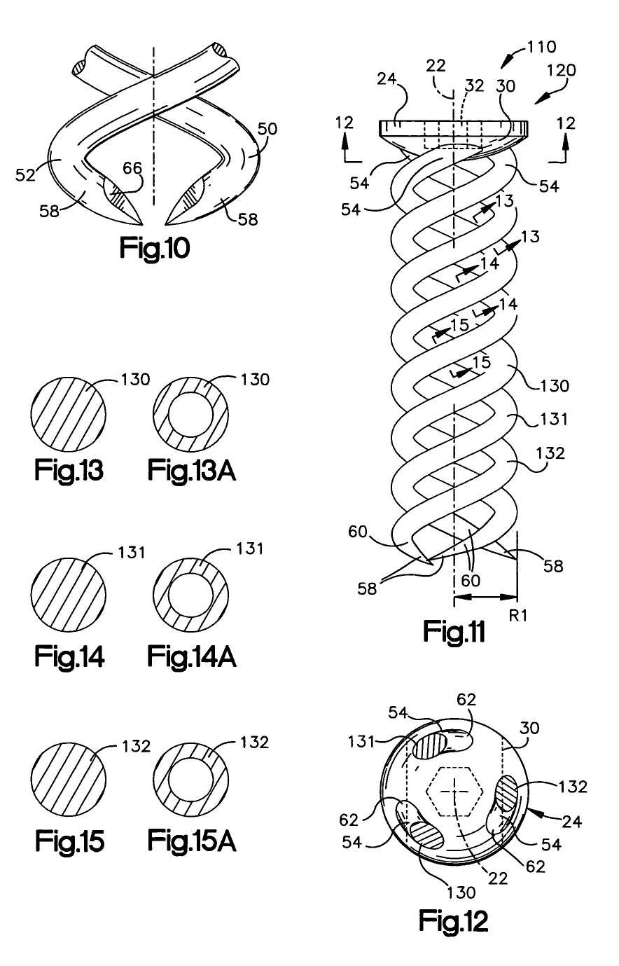

The anchor 20 is centered about a longitudinal axis 22 (FIG. 5). The anchor 20 includes a platform 24 having a cylindrical outer surface 26 extending between oppositely disposed first and second end surfaces 28 and 30 of the platform. The first end surface 28 is planar, while the second end surface 30 has a convex shape. It should be understood that the second end surface 30 could be any shape, and may be designed to be complimentary to an outer surface 19 (FIG. 1) of the S1 vertebrae.

The second end surface 30 of the platform 24 may include barbs (not shown) or other suitable structure for engaging the outer surface 19 of the S1 vertebrae. Further the second end surface 30 of the platform 24 may also be porous, pitted, or have a biocompatible surface coating, such as is shown in FIG. 7, that increases the surface area of the second end surface to promote bone in-growth and thereby assist with fixation of the anchor 20 to the S1 vertebrae.

The platform 24 further includes a hexagonal slot 32 (FIG. 5) that extends axially from the first end surface 28 toward the second end surface 30 of the platform. The hexagonal slot 32 is designed to receive a driver (not shown) for rotating the anchor 20.

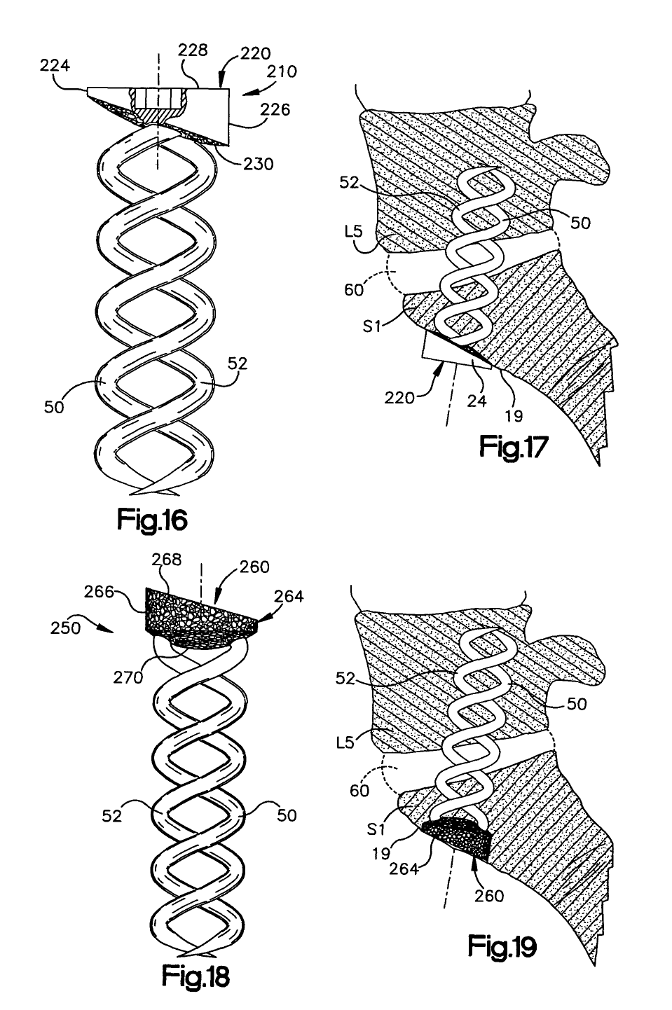

First and second helical spikes 50 and 52 project from the second end surface 30 of the platform 24. The helical spikes 50 and 52 resemble a pair of intertwined corkscrews. As shown in FIGS. 8 and 9, each of the helical spikes 50 and 52 has a solid cross-section. Alternatively, each of the helical spikes 50 and 52 could have a tubular cross-section, as illustrated in FIGS. 8A and 9A, which provides a means for matching the modulus of elasticity of the bone. It is contemplated that, with a tubular cross-section, the wall thickness can be varied/selected to match the modulus of elasticity of the bone, which can improve fixation strength and load-sharing characteristics of the anchor 20 and the bone. Further, a tubular cross-section design could provide at least a portion of the helical spikes 50 and 52 with a slightly elastic or spring-like section which would allow very limited relative movement between the L5 and S1 vertebrae.

According to the embodiment illustrated in FIGS. 1-9, the first and second helical spikes 50 and 52 extend around the axis 22. The spikes 50 and 52 extend in a helical pattern about the axis 22 at the same, constant overall radius R1 (FIG. 5). It is contemplated, however, that the first and second helical spikes 50 and 52 could extend about the axis 22 at different radiuses. Further, it is contemplated that the radius of one or both of the first and second helical spikes 50 and 52 could increase or decrease as the helical spikes extend away from the platform 24.

In order for the anchor 20 to be implanted endoscopically through a typical cannula 14 (FIG. 1), the platform 24 and the helical spikes 50 and 52 should be less than 20mm in overall diameter. It should be understood that the anchor 20 could have an overall diameter that is greater than 20 mm for certain applications, and that the anchor could be also implanted in an open surgical procedure.

In the illustrated embodiment of FIGS. 1-9, the first and second helical spikes 50 and 52 have the same axial length, and also have the same cross-sectional shape. It is contemplated, however, that the first and second helical spikes 50 and 52 could have different axial lengths. Further, it is contemplated that the helical spikes 50 and 52 could have a different cross-sectional shape, such as an oval shape. It also contemplated that the first and second helical spikes 50 and 52 could have different outer diameters (i.e., one spike being thicker than the other spike). Finally, it is contemplated that the helical spikes 50 and 52 should have the same pitch, and that the pitch of the helical spikes would be selected based on the specific surgical application and quality of the bone in which the anchor 20 is to be implanted.

Each of the first and second helical spikes 50 and 52 can be divided into three portions: a connecting portion 54, an intermediate portion 56, and a tip portion 58. The connecting portion 54 of each of the helical spikes 50 and 52 is located at a proximal end 60 that adjoins the second end surface 30 of the platform 24. The connecting portion 54 may include barbs (not shown) for resisting pull-out of the helical spikes 50 and 52 from the S1 vertebrae. According to one method for manufacturing the anchor 20, the connecting portion 54 of each of the helical spikes 50 and 52 may be fixedly attached to the platform 24 by inserting, in a tangential direction, the proximal ends 60 of the helical spikes into openings (not shown) in the second end surface 30 and welding the connecting portions 54 to the platform. The inserted proximal ends 60 of the helical spikes 50 and 52 help to reduce bending stresses on the helical spikes under tensile or shear loads.

Alternatively, the helical spikes 50 and 52 may be formed integrally with the platform 24, such as by casting the anchor 20. If the anchor 20 is cast, it is contemplated that a fillet (not shown) may be added at the junction of the helical spikes 50 and 52 and the platform 24 to strengthen the junction and minimize stress concentrations at the connecting portions 54. The fillet at the junction of the helical spikes 50 and 52 and the platform 24 also helps to reduce bending stresses in the connection portions 54 of the helical spikes under tensile or shear loads.

As best seen in FIG. 6, the connecting portions 54 at the proximal ends 60 of the first and second helical spikes 50 and 52 are spaced 180° apart about the axis 22 to balance the anchor 20 and evenly distribute loads on the helical spikes. The tip portion 58 of each of the helical spikes 50 and 52 is located at a distal end 62 of the helical spikes. The intermediate portion 56 of each of the helical spikes 50 and 52 extends between the tip portion 58 and the connecting portion 54. The intermediate portion 56 and the tip portion 58 of each of the helical spikes 50 and 52 have an outer diameter that is less than or equal to the outer diameter of the connecting portions 54. If the outer diameter of the intermediate portion 56 and the tip portion 58 is less than the outer diameter of the connecting portion 54 of each of the helical spikes 50 and 52, the increased thickness of the connecting portions will help to provide the anchor 20 with increased tensile strength at the junction of the helical spikes and the platform 24.

The tip portion 58 of each of the helical spikes 50 and 52 illustrated in FIGS. 1-9 has an elongated conical shape with a sharp pointed tip 68 for penetrating into the bones as the platform 24 of the anchor 20 is rotated in a clockwise direction. FIG. 10 illustrates an alternative, self-tapping configuration for the tip portions 58 which includes a planar surface 66 for driving into bone, in the same manner that a wood chisel turned upside-down drives into wood, as the platform 24 is rotated. It is contemplated that the tip portions 58 could also have a pyramid shape (not shown), similar to the tip of a nail.

Although the outer surfaces of the helical spikes 50 and 52 are shown as being smooth in FIGS. 1-9, it is contemplated that the outer surfaces may instead be porous, pitted, or have a biocompatible coating that increases the surface area to promote bone in-growth and thereby assist with fixation of the anchor 20 into the bone.

It is further contemplated that the tip portions 58 of the helical spikes 50 and 52 could be temporarily covered with tip protectors (not shown) to prevent accidental sticks to surgical staff and accidental damage to tissue surrounding the S1 vertebrae. Such tip protectors could be made of a bio-absorbable material, such as polylactic acid, or non-bio-absorbable material, such as medical grade silicon. The tip protectors would be manually removed or pushed-off during implantation of the anchor 20.

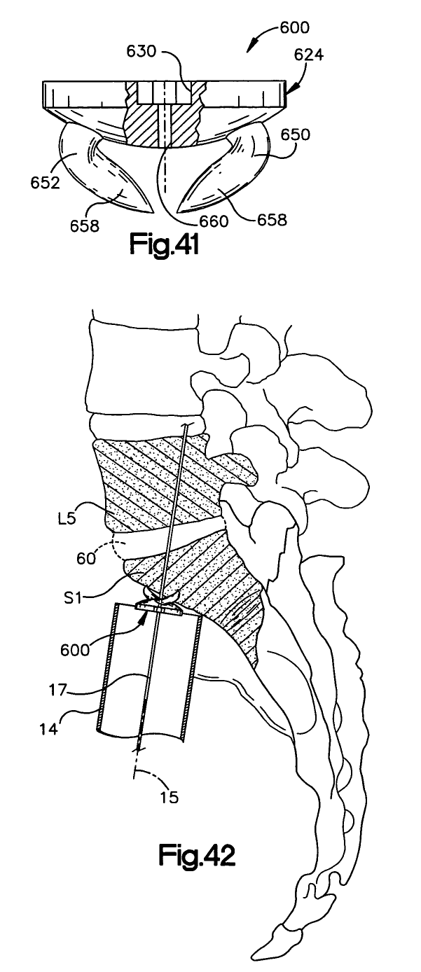

To attach the S1 vertebrae to the L5 vertebrae using the anchor 20, disk material (not shown) that normally separates the vertebrae is first removed by the surgeon. Removal of the disk material leaves an interbody space 60 (FIG. 1) between the S1 and L5 vertebrae. Next, a tool 600 (FIG. 41) may be used to punch two holes in the cortical bone of the S1 vertebrae as shown in FIG. 42. The starter tool 600 includes a platform 624 similar to the platform 24 and a plurality of helical spikes 650 and 652 similar to the helical spikes 50 and 52. The platform 624 includes a feature, such as a hexagonal drive recess 630, for drivingly rotating the starter tool 600. The spikes 650 and 652 correspond in diameter and quantity to the helical spikes 50 and 52, but are much shorter in axial length in order to increase their strength and resistance to radially outward deformation. The holes 602 and 604 are punched in locations that correspond to the spacing of the tip portions 58 of the helical spikes 50 and 52 on the anchor 20.

It should be noted that one or both of the configurations of the tip portions 58 illustrated in FIGS. 1-9 may be able to punch through the cortical bone upon rotation of the anchor 20, thus eliminating the need for the starter tool 600 to punch holes in the cortical bone.

As shown in FIG. 42, alignment of the starter tool 600 along a desired axis 15 through the S1 and L5 vertebrae may be ensured by threading the starter tool down over a wire 17 that has been previously passed through the S1 and L5 vertebrae. To allow for this, the starter tool 600 may optionally include a central bore 660 (FIG. 41).

Referring again to FIG. 1, the tip portions 58 of the helical spikes 50 and 52 of the anchor 20 are then placed in the holes in the S1 vertebrae and a rotatable driver (not shown) is inserted into the slot 32 in the platform 24. The driver is then rotated, causing the anchor 20 to rotate as well. A cylindrical sleeve 18 may be placed around the intermediate portions 56 and the connecting portions 54 of the helical spikes 50 and 52 to prevent the helical spikes from deforming radially outward during the initial rotation of the anchor 20.

Rotation of the anchor 20 screws the helical spikes 50 and 52 into the cancellous bone of the S1 vertebrae. The tangentially-oriented connection between the connecting portions 54 of the helical spikes 50 and 52 and the platform 24 minimizes bending loads on the connecting portions during rotation of the anchor 20. Further, the tangentially-oriented connection ensures that the force vector resulting from torque and axial force applied by the driver to platform 24 is transmitted along the helical centerline (not shown) of each of the helical spikes 50 and 52.

As the anchor 20 is rotated, the tip portion 58 of the first helical spike 50 penetrates the cancellous bone and cuts a first helical tunnel through the S1 vertebrae. Simultaneously, the tip portion 58 of the second helical spike 52 penetrates the cancellous bone of the S1 vertebrae and cuts a second helical tunnel. The first and second helical tunnels are shaped like the helical spikes 50 and 52, respectively.

Continued rotation of the anchor 20 embeds the helical spikes 50 and 52 deeper into the cancellous bone of the S1 vertebrae until the tip portions 58 of the helical spikes project through the upper end plate surface 21 on the S1 vertebrae and into the interbody space 60. After another rotation or so of the platform 24, the tip portions 58 of the helical spikes 50 and 52 cross through the interbody space 60 and engage the lower end plate surface 23 of the L5 vertebrae. The native ligaments (not shown) and annulus (not shown) will help to limit distraction between the L5 and S1 vertebrae. If an open surgical procedure was being used, the surgeon could stabilize the L5/S1 segment in a number of different ways to prevent overdistraction.

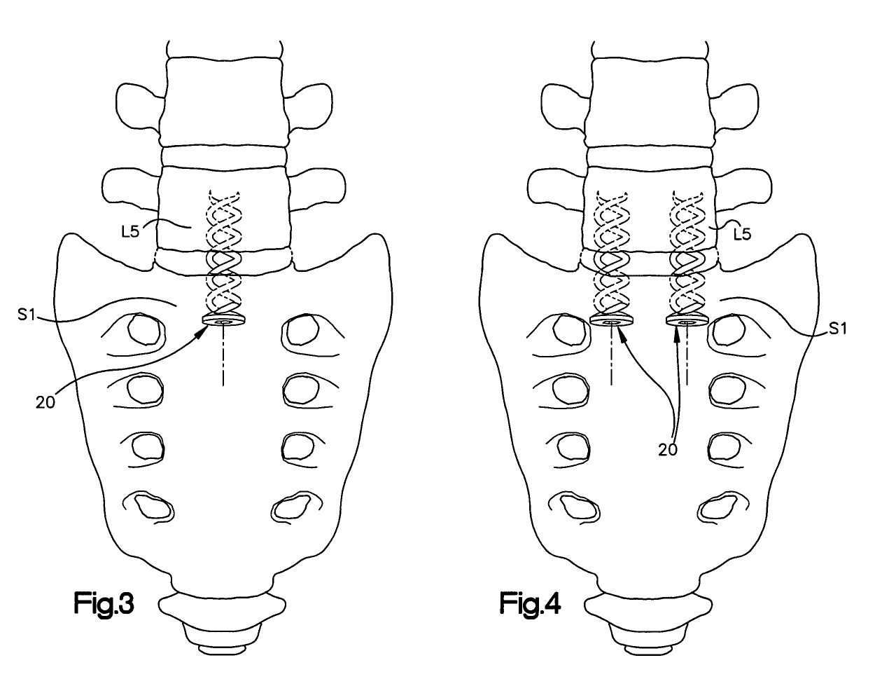

As the anchor 20 is rotated further, the first and second helical spikes 50 and 52 cut into the cancellous bone in the L5 vertebrae and extend the first and second helical tunnels 80 and 82, respectively, into the L5 vertebrae. The anchor 20 is rotated until the second end surface 30 on the platform 24 seats tightly against the outer surface 19 of the S1 vertebrae as shown in FIG. 2. FIG. 2A illustrates that the platform 24 may alternatively be recessed into the anterior surface 19 of the S1 vertebrae. In the position of FIGS. 2 and 2A, the L5 and S1 vertebrae are fixedly connected to one another by the anchor 20, yet the interbody space 60 is maintained anatomically correct. FIGS. 3 and 4 illustrate how either one or two anchors 20 can be used to connect the L5 and S1 vertebrae.

If permanent and rigid fixation of the L5 and S1 vertebrae is desired, an osteogenic (or bone graft) material 130 may be placed into the interbody space 60 as shown schematically in FIG. 2. Such material 130 may be placed via an annulotomy or a percutaneous procedure.

Because the helical spikes 50 and 52 of the anchor 20 displace much less of the cancellous bone in the L5 and S1 vertebrae during implantation than a conventional solid shank bone screw, much less torque is required to implant the anchor than is required by a conventional bone screw. Further, because the helical spikes 50 and 52 displace only a small amount of bone, the helical spikes do not create a core defect that could lead to bone deformation or failure, such as the helical spikes pulling out of the bone.

When implanted, a bone screw can be subjected to substantial forces caused by human body movement and muscle memory. In some cases, these forces can tend to pull the known screws used in such an application out of the bone or can cause the screws to toggle in the bone. However, when embedded into bones such as the L5 and S1 vertebrae shown in FIG. 2, the helical spikes 50 and 52 provide the anchor with a high resistance to pull-out forces. Further, the helical spikes 50 and 52, and their tangential connection with the platform 24, provide the anchor 20 with a high resistance to toggling in the bone. Thus, the anchor 20 provides an effective means for attaching the S1 and L5 vertebrae together to prevent relative movement while maintaining the interbody space 60. Additional advantages of the anchor 20 in the L5/S1 application include a simple one-piece construct that can be implanted in a minimally invasive procedure.

FIGS. 11-15 illustrate an apparatus 110 for attaching the L5 and S1 vertebrae in accordance with a second embodiment of the present invention. In the second embodiment of FIGS. 11-15, reference numbers that are the same as those used in the first embodiment of FIGS. 1-9 designate parts that are the same as parts in the first embodiment.

According to the second embodiment, the apparatus 110 comprises an anchor 120 having three helical spikes 130, 131, and 132 projecting tangentially from the second end surface 30 of the platform 24. As shown in FIGS. 13-15, each of the helical spikes 130-132 has a solid cross-section. Alternatively, at least a portion of each of the helical spikes 130-132 could have a tubular cross-section, as is illustrated in FIGS. 13A-15A.

As shown in FIG. 12, the connecting portions 54 at the proximal ends 60 of the helical spikes 130-132 are spaced 120° apart about the axis 22, which balances the anchor 120 and evenly distributes loads on the helical spikes. As in the first embodiment of FIGS. 1-9, in the second embodiment of FIGS. 11-15, the outer diameter of the connecting portions 54 of the helical spikes 130-132 is greater than or equal to the outer diameter of the intermediate portions 56 and the tip portions 58 of the helical spikes.

Each of the three helical spikes 130-132 extends in a helical pattern about the axis 22 at the same, constant radius R1 (FIG. 11). It is contemplated, however, that one or more of the helical spikes 130-132 could extend about the axis 22 at different radiuses. Further, it is contemplated that the radius of one or more helical spikes 130-132 could increase or decrease as the helical spikes extend away from the platform 24.

As shown in FIG. 11, the three helical spikes 130-132 have the same axial length and also have the same cross-sectional shape. It is contemplated, however, that one or more of the helical spikes 130-132 could have different axial lengths. Further, it is contemplated that one or more of the helical spikes 130-132 could have a different cross-sectional shape, such as an oval shape. It also contemplated that the one or more of the helical spikes 130-132 could have different outer diameters (i.e., one spike being thicker or thinner than the other spike(s)). Finally, it is contemplated that the helical spikes 130-132 should have the same pitch, and that the pitch of the helical spikes would be selected based on the specific surgical application and quality of the bone in which the anchor 20 is to be implanted.

The tip portion 58 of each of the helical spikes 130-132 illustrated in FIG. 11 has an elongated conical shape for penetrating into bone as the platform 24 of the anchor 120 is rotated in the clockwise direction. It should be understood that the tip portions 58 of the helical spikes 130-132 of the anchor 120 could alternatively be configured like the tip portions illustrated in FIG. 10.

Although the outer surfaces of the helical spikes 130-132 are shown as being smooth in FIGS. 11-15, it is contemplated that the outer surfaces may instead be porous, pitted, or have a biocompatible coating to assist with fixation of the anchor 120 to the L5 and S1 vertebrae.

It is further contemplated that the tip portions 58 of the helical spikes 130-132 could be temporarily covered with tip protectors (not shown) to prevent accidental sticks to surgical staff and accidental damage to tissue surrounding the fractured bone. Such tip protectors could be made of a bio-absorbable material, such as polylactic acid or a non-bio-absorbable material, such as medical grade silicon. The tip protectors would be manually removed or pushed-off during implantation of the anchor 120.

The anchor 120 according to the second embodiment of FIGS. 11-15 is implanted into the S1 vertebrae, and subsequently into the L5 vertebrae in the same manner as the anchor 20 according to the first embodiment. Because the helical spikes 130-132 of the anchor 120 displace less cancellous bone during implantation than a conventional solid shank bone screw, less torque is required to implant the anchor than is required by a conventional bone screw. Further, because the helical spikes 130-132 displace only a small amount of bone, the helical spikes do not create a core defect that could lead to bone destruction or failure, such as the spikes pulling out of the bone. When implanted in the S1 and L5 vertebrae, the anchor 120 according to the second embodiment is highly resistant to being pulled out of the bone and to toggling in the bone despite being subjected to substantial forces caused by human body movement and muscle memory. The anchor 120 thus provides an effective means for connecting the L5 and S1 vertebrae.

FIGS. 16 and 17 illustrate an apparatus 210 for attaching the L5 and S1 vertebrae in accordance with a third embodiment of the present invention. In the third embodiment of FIGS. 16 and 17, reference numbers that are the same as those used in the first embodiment of FIGS. 1-9 designate parts that are the same as parts in the first embodiment.

According to the third embodiment, the apparatus 210 comprises an anchor 220 having a platform 224. The platform 224 includes an outer surface 226 that is a wedge-shaped portion of a cylinder. The outer surface 226 extends between oppositely disposed first and second end surfaces 228 and 230 of the platform. The first and second helical spikes 50 and 52 project from the second end surface 230 of the platform 224.

The first end surface 228 is planar, while the second end surface 230 is oblique (or angled) and has a slightly convex shape. The second end surface 230 is designed to be complimentary to the anterior surface 19 of the S1 vertebrae, as may be seen in FIG. 17. The second end surface 230 of the platform 224 has a porous configuration that can be achieved in a variety of different ways, such as by spraying with a biocompatible surface coating, to increase the surface area of the end surface. Such increased surface area promotes bone in-growth and thereby assist with fixation of the anchor 220 to the S1 vertebrae.

The anchor 220 according to the third embodiment of FIGS. 16 and 17 is implanted into the S1 vertebrae, and subsequently into the L5 vertebrae, in much the same manner as the anchor 20 according to the first embodiment, the only difference being that the angled end surface 224 rests against the angled outer surface 19 of the S1 vertebrae to achieve a desirable full-surface engagement and a desired depth of screw penetration.

As discussed previously, because the helical spikes 50 and 52 displace only a small amount of bone, the helical spikes do not create a core defect that could lead to bone destruction or failure, such as the spikes pulling out of the bone. When implanted in the S1 and L5 vertebrae, the anchor 220 according to the third embodiment is highly resistant to being pulled out of the vertebrae and to toggling in the vertebrae despite being subjected to substantial forces caused by human body movement and muscle memory.

FIGS. 18 and 19 illustrate an apparatus 250 for attaching the L5 and S1 vertebrae in accordance with a fourth embodiment of the present invention. In the fourth embodiment of FIGS. 18 and 19, reference numbers that are the same as those used in the first embodiment of FIGS. 1-9 designate parts that are the same as parts in the first embodiment.

According to the fourth embodiment, the apparatus 250 comprises an anchor 260 having a platform 264. The platform 264 includes an outer surface 266 that is a wedge-shaped portion of a cylinder. The outer surface 266 extends between oppositely disposed first and second end surfaces 268 and 270 of the platform. The first and second helical spikes 50 and 52 project from the second end surface 270 of the platform 264. The second end surface 270 has convex shape, while the first end surface 268 is angled and is designed to be complimentary to the outer surface 19 of the S1 vertebrae, as may be seen in FIG. 19.

The outer surface 266 and the second end surface 270 of the platform 264 have porous configurations that can be achieved in a variety of different ways, such as by spraying with a biocompatible surface coating, to increase their respective surface areas. Such increased surface areas promote bone in-growth and thereby assist with fixation of the anchor 220 to the S1 vertebrae.

The anchor 260 according to the fourth embodiment of FIGS. 18 and 19 is implanted into the S1 vertebrae, and subsequently into the L5 vertebrae, in much the same manner as the anchor 20 according to the first embodiment, the only difference being that the platform 264 is recessed into the S1 vertebrae until the first end surface 268 is flush with the angled outer surface 19 of the S1 vertebrae to achieve a multi-surface engagement and a desired depth of screw penetration.

As discussed previously, because the helical spikes 50 and 52 displace only a small amount of bone, the helical spikes do not create a core defect that could lead to bone destruction or failure, such as the spikes pulling out of the bone. When implanted in the S1 and L5 vertebrae, the anchor 260 according to the fourth embodiment is highly resistant to being pulled out of the vertebrae and to toggling in the vertebrae despite being subjected to substantial forces caused by human body movement and muscle memory.

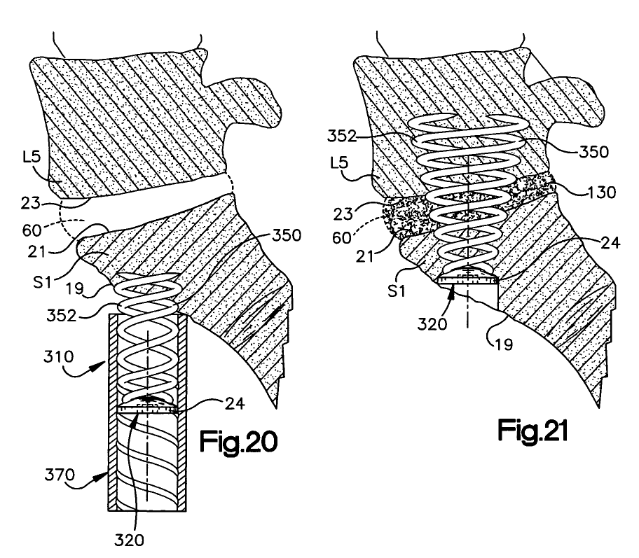

FIGS. 20-28 illustrate an apparatus 310 for attaching the L5 and S1 vertebrae in accordance with a fifth embodiment of the present invention. In the fifth embodiment of FIGS. 20-28, reference numbers that are the same as those used in the first embodiment of FIGS. 1-9 designate parts that are the same as parts in the first embodiment.

According to the fifth embodiment, the apparatus 310 comprises an anchor 320 made at least partially from a shape memory alloy that is biocompatible. As is known in the art, shape memory alloys have the ability to return to a predetermined shape when heated. When a shape memory alloy is cold, or below its transition temperature range (TTR), the material has a low yield strength and can be deformed into a new shape, which it will retain until heated. However, when a shape memory alloy is heated above its TTR, the material undergoes a change in crystal structure (from a martensite structure to an austensite structure), which causes the material to return to its original, or “memorized” shape. A memorized shape is imprinted into a shape memory alloy by first holding the material in the desired shape at a high temperature, and then continuing to hold the material in the desired shape as it cools through its TTR.

The anchor 320 of the fifth embodiment includes the platform 24 and two helical spikes 350 and 352 that are made from a shape memory alloy. As may be seen in FIG. 21, the helical spikes 350 and 352, when fully embedded in the S1 and L5 vertebrae, extend across the interbody space 60 to attach the two vertebrae.

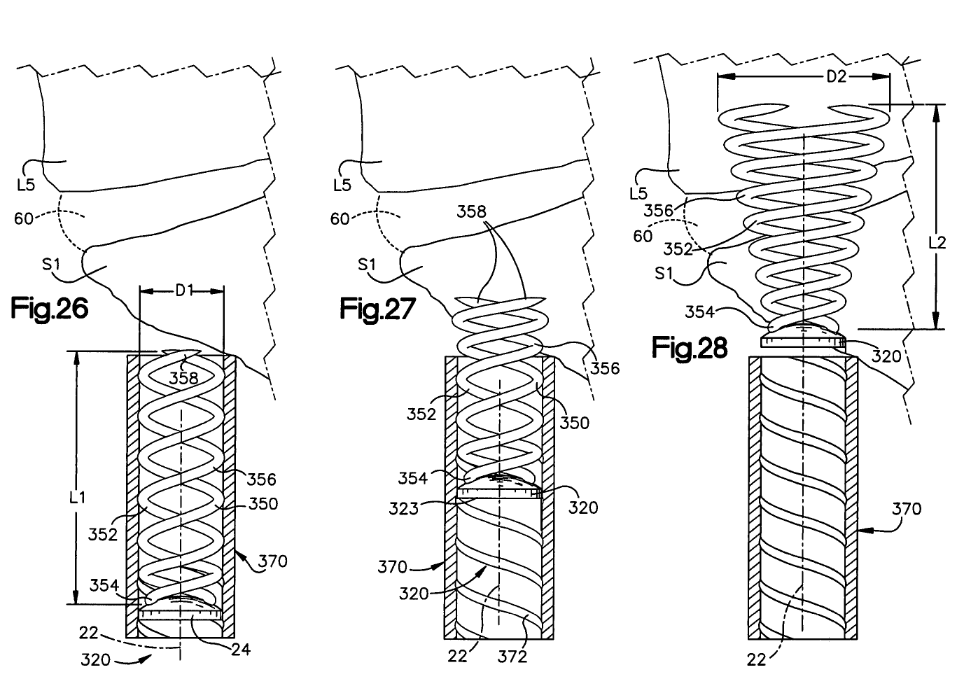

The helical spikes 350 and 352 resemble a pair of intertwined corkscrews, both of which have a conical shape that increases in diameter as the helical spikes extend away from the platform 24. As shown in FIGS. 24 and 25, each of the helical spikes 350 and 352 has a solid cross-section. Alternatively, each of the helical spikes 350 and 352 could have a tubular cross-section, as illustrated in FIGS. 24A and 25A, which provides a means for matching the modulus of elasticity of the bone.

According to the embodiment illustrated in FIGS. 20-28, the first and second helical spikes 350 and 352 extend around the axis 22. The helical spikes 350 and 352 extend symmetrically in a conical pattern about the axis 22. It is contemplated, however, that the conical shape of the first and second helical spikes 350 and 352 could be different from each other (i.e., one spike being a smaller cone than the other spike).

Each of the first and second helical spikes 350 and 352 can be divided into three portions: a connecting portion 354, an intermediate portion 356, and a tip portion 358. The connecting portion 354 of each of the helical spikes 350 and 352 is located at a proximal end 360 that adjoins the end surface 30 of the platform 24. The connecting portion 354 may include barbs (not shown) for resisting pull-out of the helical spikes 350 and 352 from the S1 vertebrae.

As mentioned previously, the helical spikes 350 and 352 of the anchor 320 are made from a shape memory alloy, which allows the anchor to have more than one shape. FIGS. 26-28 illustrate the shapes of the anchor 320 at various stages of the implantation process. The shape that is “memorized” into the material of the anchor 320 is illustrated in FIGS. 21, 22 and 28. FIG. 26 illustrates the anchor 320 in a first condition prior to implantation in the vertebrae. In the first condition, the helical spikes 350 and 352 of the anchor 320 do not have a conical shape, but instead have a generally cylindrical shape with a uniform maximum diameter D1. Further, in the first condition, the helical spikes 350 and 352 have an axial length L1. In order for the anchor 320 to take the shape of the first condition, the temperature of the anchor must be below its TTR so that the material of the anchor is soft and ductile.

The anchor 320 is moved into the first condition of FIG. 26 with the aid of a tubular sleeve 370. The sleeve 370 is made from a hard metal and includes internal threads 372 (FIG. 27) for mating with the helical spikes 350 and 352 of the anchor 320 to aid in drawing the helical spikes into the sleeve upon rotation of the anchor. With the temperature of the anchor 320 below its TTR, the anchor is pulled into the sleeve 370 by rotating the platform 24 in a first direction with a driver (not shown) that fits into the slot 32. As the helical spikes 350 and 352 are drawn into the sleeve 370, the helical spikes are compressed radially inward, causing their axial length to grow to the axial length L1.

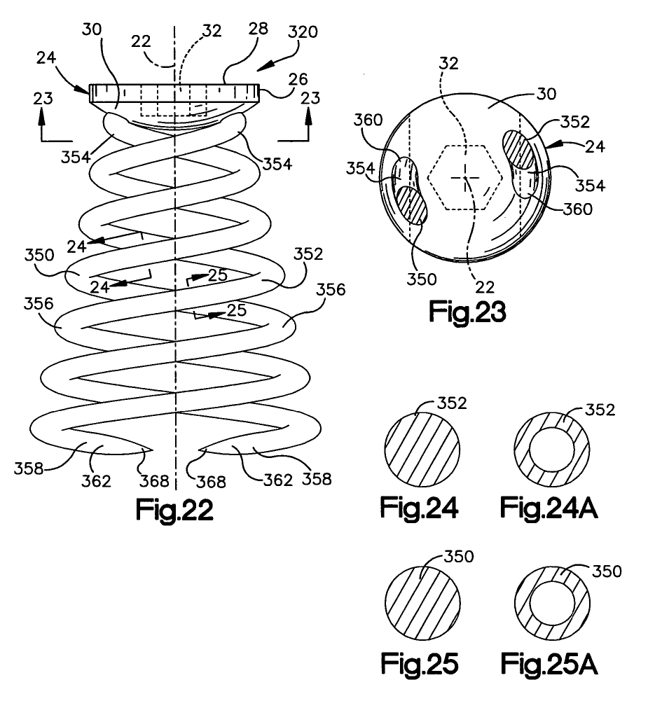

FIG. 27 illustrates the anchor 320 during implantation into the S1 vertebrae. As shown in FIG. 27, the helical spikes 350 and 352 emerge from the sleeve 370 when the platform 24 is rotated in a second direction that is opposite the first direction. As the helical spikes 350 and 352 emerge from the sleeve 370, it is desired that the helical spikes return to the memorized conical shape of FIG. 22. To return the helical spikes 350 and 352 to the conical shape as they emerge from the sleeve 370, heat is applied to the anchor 320 until the temperature of the anchor exceeds the TTR for the shape memory material. Simple body temperature may be sufficient to raise the temperature of the anchor 320 above its TTR. If additional heat is needed, heat may be applied in many ways, such as passing electric current through a wire connected with the anchor 320 or the sleeve 370, transmitting radio waves that inductively heat the anchor, or applying a hot saline pack to the sleeve.

With the helical spikes 350 and 352 expanding radially, but contracting axially, as they emerge from the sleeve 370, the helical spikes are implanted into the S1 vertebrae, and subsequently into the L5 vertebrae, in the conical shape, or second condition, illustrated in FIG. 28. As shown in FIG. 28, in the implanted second condition, the helical spikes 350 and 352 have a maximum diameter D2 that is larger than the maximum diameter D1 of the helical spikes in the first condition. Further, in the implanted second condition, the helical spikes 350 and 352 have an axial length L2 that is smaller than the axial length of the helical spikes in the first condition.

As is illustrated in FIGS. 26-28, the anchor 320 is implanted in the S1 and L5 vertebrae in the same basic manner that the anchor 20 was implanted, except that the shape of the helical spikes 350 and 352 changes during implantation, as described above, since heat being applied to the anchor 320 until the temperature of the anchor exceeds the TTR for the shape memory material. The initial rotation of the anchor 320 screws the helical spikes 350 and 352 into the cancellous bone of the S1 vertebrae. Continued rotation of the anchor 320 embeds the helical spikes 350 and 352 deeper into the cancellous bone of the S1 vertebrae until the tip portions 58 of the helical spikes project through the upper end plate surface 21 and into the interbody space 60.

After another rotation or so of the platform 24, the tip portions 58 of the helical spikes 350 and 352 cross through the interbody space 60 and engage the lower end plate surface 23 of the L5 vertebrae. The native ligaments (not shown) and annulus (not shown) will help to limit distraction between the L5 and S1 vertebrae. If an open surgical procedure was being used, the surgeon could stabilize the L5/S1 segment in a number of different ways to prevent overdistraction. Further rotation of the platform 24 causes the tip portions 58 of the helical spikes 350 and 352 to penetrate into the cancellous bone of the L5 vertebrae. The anchor 320 is rotated until the platform 24 seats is recessed into the S1 vertebrae as shown in FIG. 21.

By the time the platform 24 is recessed into the outer surface 19 of the S1 vertebrae, the helical spikes 350 and 352 are fully hardened and have nearly completed their shift into their memorized shape. With the S1 and L5 vertebrae attached to each other by the anchor 320, an osteogenic (or bone graft) material 130 (FIG. 21) can then be placed into the interbody space 60 to achieve permanent and rigid fixation of the L5 and S1 vertebrae. Such material 130 may be placed via an annulotomy or a percutaneous procedure.

As previously discussed with regard to the first embodiment, because the helical spikes 350 and 352 of the anchor 320 displace less bone in the S1 and L5 vertebrae during implantation than a conventional solid shank bone screw, less torque is required to implant the anchor than is required by a conventional bone screw. Further, the helical spikes 350 and 352 do not create a core defect that could lead to bone deformation or failure, such as the helical spikes pulling out of the bone. Also, when implanted, the anchor 320 is highly resistant to being pulled axially from the bone and to toggling within the bone.

In addition, the conical shape of the helical spikes 350 and 352 advantageously increases the amount of surface area engaged by the anchor 320, which spreads any load on the anchor out over different areas of the L5 and S1 vertebrae and provides fixation over a larger volume of bone. This advantage of the conical shape of the helical spikes 350 and 352 is especially helpful when implanting the anchor 320 in osteoporotic bone.

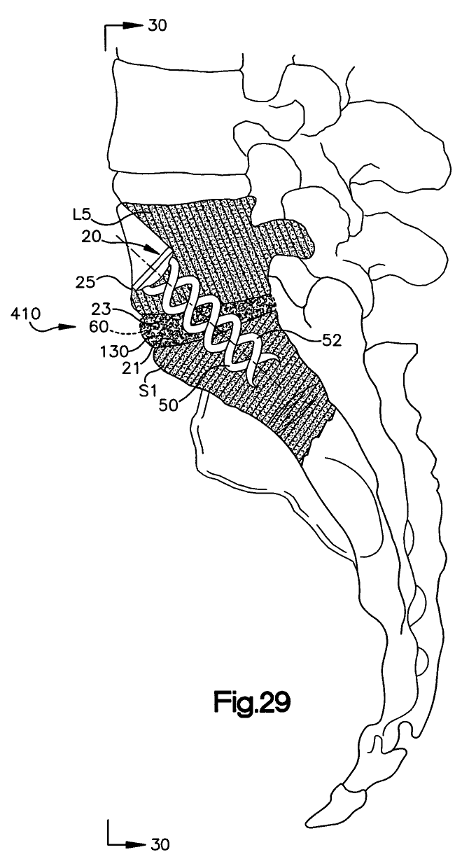

FIGS. 29-31 illustrate an apparatus 410 for attaching the L5 and S1 vertebrae in accordance with a sixth embodiment of the present invention. In the sixth embodiment of FIGS. 29-31, reference numbers that are the same as those used in the first embodiment of FIGS. 1-9 designate parts that are the same as parts in the first embodiment.

According to the sixth embodiment, the apparatus 410 comprises the anchor 20 described in the first embodiment above, but the anchor is implanted in a different manner. As may be seen in FIG. 29, the anchor 20 is screwed into the L5 vertebrae first, and then into the S1 vertebrae.

Using the method of the sixth embodiment to attach the L5 vertebrae to the S1 vertebrae, disk material (not shown) that normally separates the vertebrae is first removed by the surgeon. Removal of the disk material leaves an interbody space 60 (FIG. 1) between the S1 and L5 vertebrae. Next, the tool 600 (FIG. 41) may be used to punch two holes in the cortical bone in the anterior surface 25 of the L5 vertebrae. It should be noted that one or both of the configurations of the tip portions 58 illustrated in FIGS. 1-9 and 29-31 may be able to punch through the cortical bone upon rotation of the anchor 20, thus eliminating the need for the starter tool 600 to punch holes in the cortical bone. Alignment of the starter tool 600 along a desired axis through the L5 and S1 vertebrae may be ensured by threading the starter tool down over a wire (not shown) that has been previously passed through the L5 and S1 vertebrae. To allow for this, the starter tool 600 may include the central bore 660 (FIG. 41).

The tip portions 58 are then placed in the holes in the anterior surface 25 of the L5 vertebrae and a rotatable driver (not shown) is inserted into the slot 32 in the platform 24. The driver is then rotated, causing the anchor 20 to rotate as well. A cylindrical sleeve, such as the sleeve 18 in FIG. 1, may be placed around the intermediate portions 56 and the connecting portions 54 of the helical spikes 50 and 52 to prevent the helical spikes from deforming radially outward during the initial rotation of the anchor 20.

Rotation of the anchor 20 screws the helical spikes 50 and 52 into the cancellous bone of the L5 vertebrae. The tangentially-oriented connection between the connecting portions 54 of the helical spikes 50 and 52 and the platform 24 minimizes bending loads on the connecting portions during rotation of the anchor 20. Further, the tangentially-oriented connection ensures that the force vector resulting from torque and axial force applied by the driver to platform 24 is transmitted along the helical centerline (not shown) of each of the helical spikes 50 and 52.

As the anchor 20 is rotated, the tip portion 58 of the first helical spike 50 penetrates the cancellous bone and cuts a first helical tunnel through the L5 vertebrae. Simultaneously, the tip portion 58 of the second helical spike 52 penetrates the cancellous bone of the L5 vertebrae and cuts a second helical tunnel. The first and second helical tunnels are shaped like the helical spikes 50 and 52, respectively.

Continued rotation of the anchor 20 embeds the helical spikes 50 and 52 deeper into the cancellous bone of the L5 vertebrae until the tip portions 58 of the helical spikes project through the lower end plate surface 23 on the L5 vertebrae and into the interbody space 60. After another rotation or so of the platform 24, the tip portions 58 of the helical spikes 50 and 52 cross through the interbody space 60 and engage the upper end plate surface 21 of the S1 vertebrae. The native ligaments (not shown) and annulus (not shown) will help to limit distraction between the L5 and S1 vertebrae. If an open surgical procedure was being used, the surgeon could stabilize the L5/S1 segment in a number of different ways to prevent overdistraction.

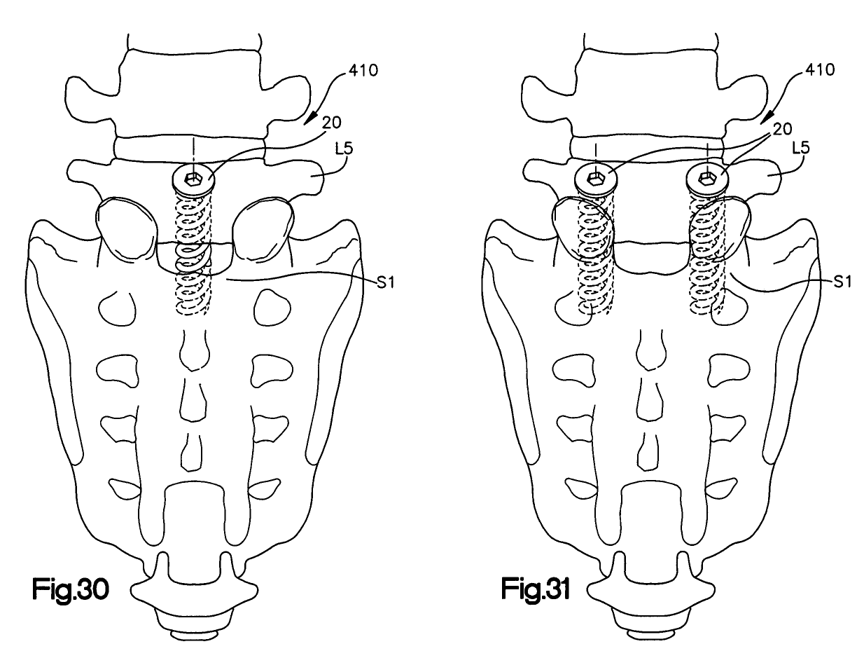

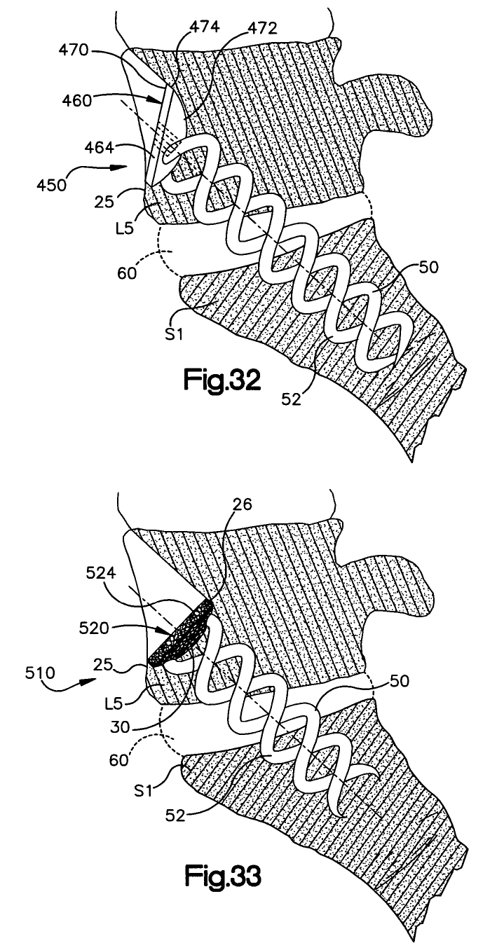

As the anchor 20 is rotated further, the first and second helical spikes 50 and 52 cut into the cancellous bone in the S1 vertebrae and extend the first and second helical tunnels, respectively, into the S1 vertebrae. The anchor 20 is rotated until the second end surface 30 on the platform 24 is recessed into the anterior surface 25 of the L5 vertebrae as shown in FIG. 29. In this position, the L5 and S1 vertebrae are fixedly connected to one another by the anchor 20, yet the interbody space 60 is maintained anatomically correct. FIGS. 30 and 31 illustrate how either one or two anchors 20 can be used to connect the L5 and S1 vertebrae according to the apparatus 410 of the sixth embodiment.

If permanent and rigid fixation of the L5 and S1 vertebrae is desired, an osteogenic (or bone graft) material 130 may be placed into the interbody space 60 as shown schematically in FIG. 29. Such material 130 may be placed via an annulotomy or a percutaneous procedure.

Because the helical spikes 50 and 52 of the anchor 20 displace much less of the cancellous bone in the L5 and S1 vertebrae during implantation than a conventional solid shank bone screw, much less torque is required to implant the anchor than is required by a conventional bone screw. Further, because the helical spikes 50 and 52 displace only a small amount of bone, the helical spikes do not create a core defect that could lead to bone deformation or failure, such as the helical spikes pulling out of the bone.

When implanted, a bone screw can be subjected to substantial forces caused by human body movement and muscle memory. In some cases, these forces can tend to pull the known screws used in such an application out of the bone or can cause the screws to toggle in the bone. However, when embedded into bones such as the L5 and S1 vertebrae shown in FIG. 29, the helical spikes 50 and 52 provide the anchor with a high resistance to pull-out forces. Further, the helical spikes 50 and 52, and their tangential connection with the platform 24, provide the anchor 20 with a high resistance to toggling in the bone. Thus, the anchor 20 provides an effective means for attaching the S1 and L5 vertebrae together to prevent relative movement while maintaining the interbody space 60. Additional advantages of the anchor 20 in the L5/S1 application include a simple one-piece construct that can be implanted in a minimally invasive procedure.

FIG. 32 illustrates an apparatus 450 for attaching the L5 and S1 vertebrae in accordance with a seventh embodiment of the present invention. In the seventh

relatively movable first and second tubular members, said first tubular member being at least partially disposed coaxially within said second tubular member, said first tubular member having a distal end and a plurality of infusion ports adjacent said distal end for directing the flow of a thrombolytic liquid, said plurality of infusion ports being spaced axially apart, said plurality of infusion ports varying in size and increasing in diameter toward said distal end to provide an evenly distributed flow pattern throughout said infusion section, at least a portion of said plurality of infusion ports being coverable by said second tubular member; and

an infusion section having an axial length that is variable, said infusion section being defined by an uncovered portion of said plurality of infusion ports, said axial length of said infusion section being varied by relative movement between said first and second tubular members which changes the quantity of said infusion ports in said uncovered portion of said

plurality of infusion ports. embodiment of FIG. 32, reference numbers that are the same as those used in the first embodiment of FIGS. 1-9 designate parts that are the same as parts in the first embodiment.

According to the seventh embodiment, the apparatus 450 comprises an anchor 460 having a platform 464 and the helical spikes 50 and 52. The platform 464 has a semi-egg shape defined by an oblique first end surface 470, an arcuate second end surface 472, and a cylindrical surface 474 extending between the end surfaces.

In the seventh embodiment of FIG. 32, the first and second helical spikes 50 and 52 have the same axial length, and also have the same cross-sectional shape. It is contemplated, however, that the first and second helical spikes 50 and 52 could have different axial lengths. Further, it is contemplated that the helical spikes 50 and 52 could have a different cross-sectional shape, such as an oval shape. It also contemplated that the first and second helical spikes 50 and 52 could have different outer diameters (i.e., one spike being thicker than the other spike). Finally, it is contemplated that the helical spikes 50 and 52 should have the same pitch, and that the pitch of the helical spikes would be selected based on the specific surgical application and quality of the bone in which the anchor 460 is to be implanted.

The anchor 460 is implanted in the same basic manner as the anchor 20 in the sixth embodiment of FIGS. 29-31 and functions in the same manner to attach the L5 and S1 vertebrae while maintaining the interbody space 60. The semi-egg-shaped design of the platform 464 minimizes the amount of bone displaced by recessing the platform of the anchor 460 into the anterior surface 25 of the L5 vertebrae.

FIG. 33 illustrates an apparatus 510 for attaching the L5 and S1 vertebrae in accordance with an eighth embodiment of the present invention. In the eighth embodiment of FIG. 33, reference numbers that are the same as those used in the first embodiment of FIGS. 1-9 designate parts that are the same as parts in the first embodiment.

According to the eighth embodiment, the apparatus 510 comprises an anchor 520 having a platform 524 and a pair of helical spikes 50 and 52. The platform 524 is similar to the platform 24 of the first embodiment and includes the outer surface 26 and the second end surface 30. The outer surface 26 and the second end surface 30 on the platform 524 have porous configurations that can be achieved in a variety of different ways, such as by spraying with a biocompatible surface coating, to increase their respective surface areas.

The anchor 520 is implanted in the same basic manner as the anchor 20 in the sixth embodiment of FIGS. 29-31 and functions in the same manner to attach the L5 and S1 vertebrae while maintaining the interbody space 60. The increased surface areas on the outer surface 26 and the second end surface 30 on the platform 524 promote bone in-growth and thereby assist with fixation of the anchor 520 to the L5 vertebrae.

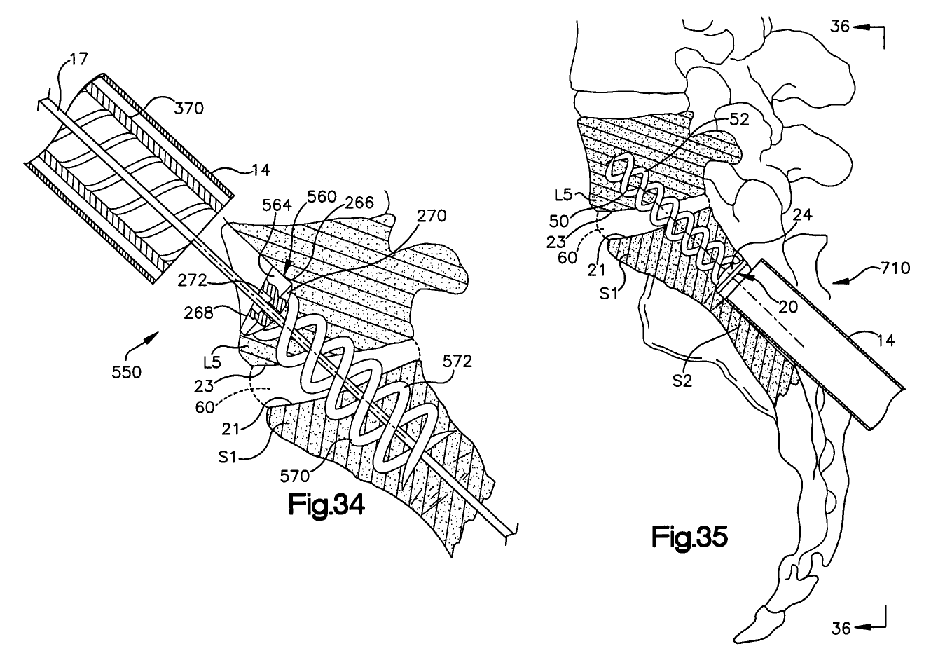

FIG. 34 illustrates an apparatus 550 for attaching the L5 and S1 vertebrae in accordance with a ninth embodiment of the present invention. In the ninth embodiment of FIG. 34, reference numbers that are the same as those used in the first embodiment of FIGS. 1-9 designate parts that are the same as parts in the first embodiment.

According to the ninth embodiment, the apparatus 550 comprises an anchor 560 having a platform 564 and a pair of helical spikes 570 and 572. The platform 564 is similar to the platform 264 of the fourth embodiment of FIG. 18 and includes the outer surface 266, the oblique first end surface 268, and the second end surface 270. The platform 564 further includes a hexagonal recess 272 for receiving a driver (not shown), and a passage (not shown) located on the axis 22 for receiving a wire 17 for aligning the anchor 560.

The helical spikes 570 and 572 are identical to the helical spikes 350 and 352 of the fifth embodiment and are thus made of a shape memory alloy. The anchor 560 is implanted in a similar manner to the anchor 20 in the sixth embodiment of FIGS. 29-31, except that the shape of the helical spikes 570 and 572 changes during implantation, as described with regard to the fifth embodiment of FIGS. 20-28, since heat is applied to the anchor 560 until the temperature of the anchor exceeds the TTR for the shape memory material. The initial rotation of the anchor 550 screws the helical spikes 570 and 572 into the cancellous bone of the L5 vertebrae. Continued rotation of the anchor 560 embeds the helical spikes 570 and 572 deeper into the cancellous bone of the L5 vertebrae until the tip portions of the helical spikes project through the lower end plate surface 23 and into the interbody space 60.

After another rotation or so of the platform 564, the tip portions of the helical spikes 570 and 572 cross through the interbody space 60 and engage the upper end plate surface 21 of the S1 vertebrae. Further rotation of the platform 564 causes the tip portions of the helical spikes 570 and 572 to penetrate into the cancellous bone of the S1 vertebrae. The anchor 550 is rotated until the platform 564 is recessed into the L5 vertebrae as shown in FIG. 34.

By the time the platform 564 is recessed into the anterior surface of the L5 vertebrae, the helical spikes 570 and 572 are fully hardened and have nearly completed their shift into their memorized shape. With the S1 and L5 vertebrae attached to each other by the anchor 550, an osteogenic (or bone graft) material can then be placed into the interbody space 60 to achieve permanent and rigid fixation of the L5 and S1 vertebrae.

As previously discussed, because the helical spikes 570 and 572 of the anchor 560 displace less bone in the S1 and L5 vertebrae during implantation than a conventional solid shank bone screw, less torque is required to implant the anchor than is required by a conventional bone screw. Further, the helical spikes 570 and 572 do not create a core defect that could lead to bone deformation or failure, such as the helical spikes pulling out of the bone. Also, when implanted, the anchor 550 is highly resistant to being pulled axially from the bone and to toggling within the bone.

In addition, the conical shape of the helical spikes 570 and 572 advantageously increases the amount of surface area engaged by the anchor 560, which spreads any load on the anchor out over different areas of the L5 and S1 vertebrae and provides fixation over a larger volume of bone. This advantage of the conical shape of the helical spikes 570 and 572 is especially helpful when implanting the anchor 560 in osteoporotic bone.

FIGS. 35-37 illustrate an apparatus 710 for attaching the L5 and S1 vertebrae in accordance with a tenth embodiment of the present invention. In the tenth embodiment of FIGS. 35-37, reference numbers that are the same as those used in the first embodiment of FIGS. 1-9 designate parts that are the same as parts in the first embodiment.

According to the tenth embodiment, the apparatus 710 comprises the anchor 20 previously described, although the first and second helical spikes 50 and 52 may be slightly longer than illustrated in the earlier embodiments because the spikes need to extend across a longer distance. To attach the S1 vertebrae to the L5 vertebrae using the anchor 20, disk material (not shown) that normally separates the vertebrae is first removed by the surgeon. Removal of the disk material leaves an interbody space 60 between the S1 and L5 vertebrae. A posterior laminectomy is then performed to gain access to the S1 vertebrae via the S2 vertebrae.

Next, the tool 600 (FIG. 41) may be used to punch two holes in the cortical bone of the S1 vertebrae. Alignment of the starter tool 600 along a desired axis through the S1 and L5 vertebrae may be ensured by threading the starter tool down over a wire (not shown) that has been previously passed through the S1 and L5 vertebrae.

The tip portions 58 are then placed in the holes in the S1 vertebrae and a rotatable driver (not shown) is inserted into the slot 32 in the platform 24. The driver is then rotated, causing the anchor 20 to rotate as well. Rotation of the anchor 20 screws the helical spikes 50 and 52 into the cancellous bone of the S1 vertebrae. The tangentially-oriented connection between the connecting portions 54 of the helical spikes 50 and 52 and the platform 24 minimizes bending loads on the connecting portions during rotation of the anchor 20. Further, the tangentially-oriented connection ensures that the force vector resulting from torque and axial force applied by the driver to platform 24 is transmitted along the helical centerline (not shown) of each of the helical spikes 50 and 52.

As the anchor 20 is rotated, the tip portion 58 of the first helical spike 50 penetrates the cancellous bone and cuts a first helical tunnel through the S1 vertebrae. Simultaneously, the tip portion 58 of the second helical spike 52 penetrates the cancellous bone of the S1 vertebrae and cuts a second helical tunnel. The first and second helical tunnels are shaped like the helical spikes 50 and 52, respectively.

Continued rotation of the anchor 20 embeds the helical spikes 50 and 52 deeper into the cancellous bone of the S1 vertebrae until the tip portions 58 of the helical spikes project through the upper end plate surface 21 on the S1 vertebrae and into the interbody space 60. After another rotation or so of the platform 24, the tip portions 58 of the helical spikes 50 and 52 cross through the interbody space 60 and engage the lower end plate surface 23 of the L5 vertebrae. The native ligaments (not shown) and annulus (not shown) will help to limit distraction between the L5 and S1 vertebrae. If an open surgical procedure was being used, the surgeon could stabilize the L5/S1 segment in a number of different ways to prevent overdistraction.

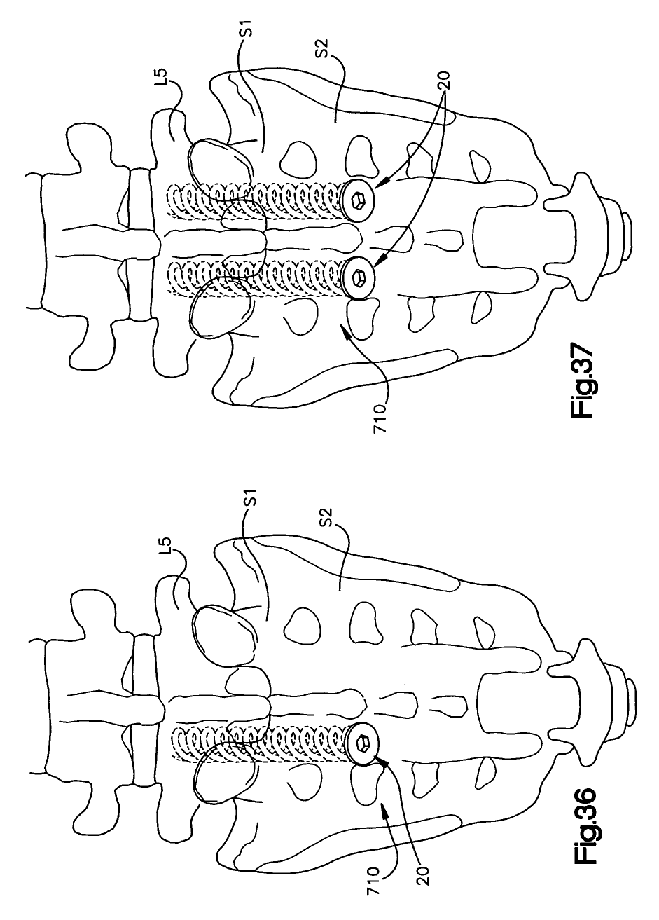

As the anchor 20 is rotated further, the first and second helical spikes 50 and 52 cut into the cancellous bone in the L5 vertebrae and extend the first and second helical tunnels into the L5 vertebrae. The anchor 20 is rotated until the platform 24 is recessed to a desired location in the S1 vertebrae as shown in FIG. 35. In this position, the L5 and S1 vertebrae are fixedly connected to one another by the anchor 20, yet the interbody space 60 is maintained anatomically correct. FIGS. 36 and 37 illustrate how either one or two anchors 20 can be used to connect the L5 and S1 vertebrae according to the apparatus 710. If permanent and rigid fixation of the L5 and S1 vertebrae is desired, an osteogenic (or bone graft) material (not shown) may be placed into the interbody space 60 as shown schematically in FIG. 35.

Because the helical spikes 50 and 52 of the anchor 20 displace much less of the cancellous bone in the L5 and S1 vertebrae during implantation than a conventional solid shank bone screw, much less torque is required to implant the anchor than is required by a conventional bone screw. Further, because the helical spikes 50 and 52 displace only a small amount of bone, the helical spikes do not create a core defect that could lead to bone deformation or failure, such as the helical spikes pulling out of the bone.

When implanted, a bone screw can be subjected to substantial forces caused by human body movement and muscle memory. In some cases, these forces can tend to pull the known screws used in such an application out of the bone or can cause the screws to toggle in the bone. However, when embedded into bones such as the L5 and S1 vertebrae shown in FIG. 35, the helical spikes 50 and 52 provide the anchor with a high resistance to pull-out forces. Further, the helical spikes 50 and 52 provide the anchor 20 with a high resistance to toggling in the bone. Thus, the anchor 20 provides an effective means for attaching the S1 and L5 vertebrae together to prevent relative movement while maintaining the interbody space 60.

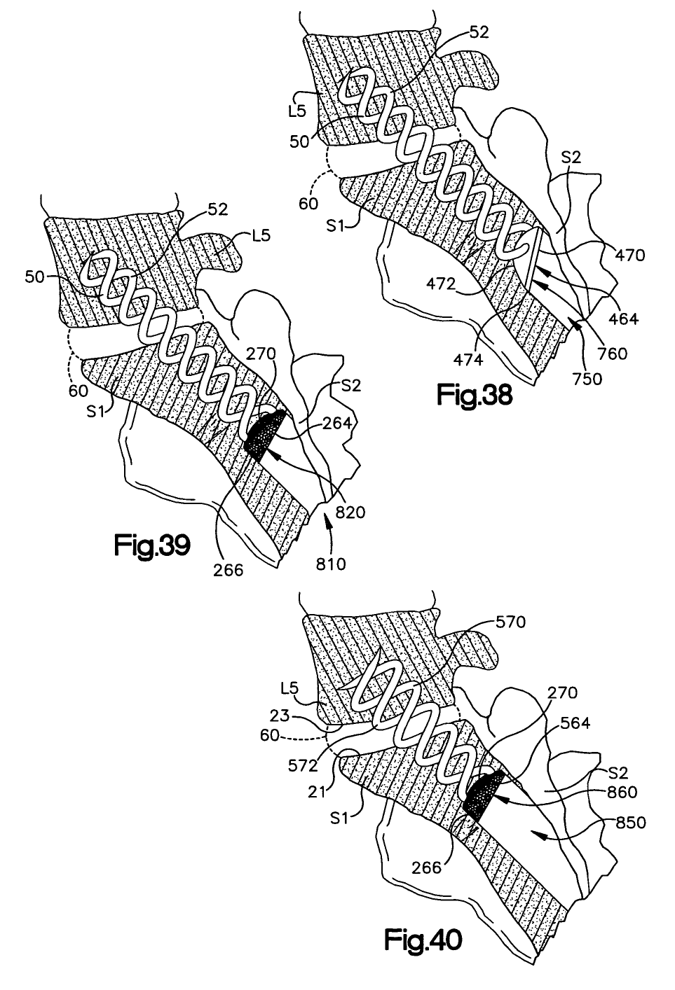

FIG. 38 illustrates an apparatus 750 for attaching the L5 and S1 vertebrae in accordance with an eleventh embodiment of the present invention. In the eleventh embodiment of FIG. 38, reference numbers that are the same as those used in the first embodiment of FIGS. 1-9 designate parts that are the same as parts in the first embodiment.

According to the eleventh embodiment, the apparatus 750 comprises an anchor 760 that is very similar to the anchor 460 discussed in the seventh embodiment of FIG. 32 and includes the platform 464 and the helical spikes 50 and 52. The platform 464 has a semi-egg shape defined by the oblique first end surface 470, the arcuate second end surface 472, and the cylindrical surface 474 extending between the end surfaces.

In the eleventh embodiment of FIG. 38, the first and second helical spikes 50 and 52 have the same axial length, and also have the same cross-sectional shape. It is contemplated, however, that the first and second helical spikes 50 and 52 could have different axial lengths. Further, it is contemplated that the helical spikes 50 and 52 could have a different cross-sectional shape, such as an oval shape. It also contemplated that the first and second helical spikes 50 and 52 could have different outer diameters (i.e., one spike being thicker than the other spike). Finally, it is contemplated that the helical spikes 50 and 52 should have the same pitch, and that the pitch of the helical spikes would be selected based on the specific surgical application and quality of the bone in which the anchor 760 is to be implanted.

The anchor 760 is implanted in the same basic manner as the anchor 20 in the tenth embodiment of FIGS. 35-37 and functions in the same manner to attach the L5 and S1 vertebrae while maintaining the interbody space 60. The semi-egg-shaped design of the platform 464 minimizes the amount of bone displaced by recessing the platform of the anchor 760 into the posterior surface of the sacrum.

FIG. 39 illustrates an apparatus 810 for attaching the L5 and S1 vertebrae in accordance with an twelfth embodiment of the present invention. In the twelfth embodiment of FIG. 39, reference numbers that are the same as those used in the first embodiment of FIGS. 1-9 designate parts that are the same as parts in the first embodiment.

According to the twelfth embodiment, the apparatus 810 comprises an anchor 820 that is identical to the anchor 260 of the fourth embodiment of FIG. 18. The anchor 820 this includes the platform 264 and helical spikes 50 and 52. The platform 264 includes the outer surface 266 and the second end surface 270. The outer surface 266 and the second end surface 270 on the platform 264 have porous configurations that can be achieved in a variety of different ways, such as by spraying with a biocompatible surface coating, to increase their respective surface areas.

The anchor 820 is implanted in the same basic manner as the anchor 20 in the tenth embodiment of FIGS. 35-37 and functions in the same manner to attach the L5 and S1 vertebrae while maintaining the interbody space 60. The increased surface areas on the outer surface 266 and the second end surface 270 on the platform 264 promote bone in-growth and thereby assist with fixation of the anchor 820 to the sacrum.

FIG. 40 illustrates an apparatus 850 for attaching the L5 and S1 vertebrae in accordance with a thirteenth embodiment of the present invention. In the thirteenth embodiment of FIG. 40, reference numbers that are the same as those used in the first embodiment of FIGS. 1-9 designate parts that are the same as parts in the first embodiment.

According to the thirteenth embodiment, the apparatus 850 comprises an anchor 860 that is similar to the anchor 560 in the ninth embodiment of FIG. 34 and thus includes the platform 564 and helical spikes 570 and 572. The platform 564 is similar to the platform 264 of the fourth embodiment of FIG. 18 and includes the outer surface 266, the oblique first end surface 268, and the second end surface 270. The platform 564 further includes the hexagonal recess (not shown) for receiving a driver.

The helical spikes 570 and 572 are identical to the helical spikes in the ninth embodiment and are thus made of a shape memory alloy. The anchor 750 is implanted in a similar manner to the anchor 820 in the twelfth embodiment of FIG. 39, except that the shape of the helical spikes 570 and 572 changes during implantation, as described previously, since heat is applied to the anchor 860 until the temperature of the anchor exceeds the TTR for the shape memory material. The initial rotation of the anchor 860 screws the helical spikes 570 and 572 into the cancellous bone of the S1 vertebrae. Continued rotation of the anchor 860 embeds the helical spikes 570 and 572 deeper into the cancellous bone of the S1 vertebrae until the tip portions 58 of the helical spikes project through the upper end plate surface 21 and into the interbody space 60.

After another rotation or so of the platform 564, the tip portions 58 of the helical spikes 570 and 572 cross through the interbody space 60 and engage the lower end plate surface 23 of the L5 vertebrae. Further rotation of the platform 564 causes the tip portions 58 of the helical spikes 570 and 572 to penetrate into the cancellous bone of the L5 vertebrae. The anchor 860 is rotated until the platform 564 is recessed into the S1 vertebrae as shown in FIG. 40.

By the time the platform 564 is recessed into the posterior surface of the S1 vertebrae, the helical spikes 570 and 572 are fully hardened and have nearly completed their shift into their memorized shape. With the S1 and L5 vertebrae attached to each other by the anchor 860, an osteogenic (or bone graft) material (not shown) can then be placed into the interbody space 60 to achieve permanent and rigid fixation of the L5 and S1 vertebrae.

As previously discussed, because the helical spikes 570 and 572 of the anchor 860 displace less bone in the S1 and L5 vertebrae during implantation than a conventional solid shank bone screw, less torque is required to implant the anchor than is required by a conventional bone screw. Further, the helical spikes 570 and 572 do not create a core defect that could lead to bone deformation or failure, such as the helical spikes pulling out of the bone. Also, when implanted, the anchor 860 is highly resistant to being pulled axially from the bone and to toggling within the bone.

In addition, the conical shape of the helical spikes 570 and 572 advantageously increases the amount of surface area engaged by the anchor 860, which spreads any load on the anchor out over different areas of the L5 and S1 vertebrae and provides fixation over a larger volume of bone. This advantage of the conical shape of the helical spikes 570 and 572 is especially helpful when implanting the anchor 860 in osteoporotic bone.

From the above description of the invention, those skilled in the art will perceive improvements, changes and modifications. Such improvements, changes and modifications within the skill of the art are intended to be covered by the appended claims.

Having described the invention, I claim:

1. An apparatus for attaching a first bone to an adjacent second bone, the second bone being separated from the first bone by a space between the bone, said apparatus comprising:
an anchor having a platform for drivingly rotating said anchor and at least two helical spikes for embedding into at least one of the first and second bones upon rotation of said platform, said platform having a first surface that extends generally transverse to a longitudinal axis of said anchor;
said at least two helical spikes projecting from said first surface of said platform and extending around said longitudinal axis, said at least two helical spikes having a tip portion at a distal end which penetrates into bone as said platform is rotated;
said anchor having a first condition in which a first portion of each of said at least two helical spikes is extendable into one of the first and second bones, said anchor further having a second condition in which said first portions are extendable into the other of the first and second bones and a second portion of each of said at least two helical spikes is extendable into said one bone to attach the first and second bones to one another while maintaining the space between the bones;
each of said at least two helical spikes further including a third portion extending between said first and second portions and that, when said anchor is embedded into the first and second bones, extends across the space between the bones.

2. The apparatus of claim 1 wherein in said first condition of said anchor, said at least two helical spikes are for embedding into one of the first and second bones and, in said second condition of said anchor, said at least two helical spikes are for embedding into both of the first and second bones.

3. The apparatus of claim 1 wherein each of said at least two helical spikes, when implanted, has a conical shape that increases in diameter as said at least two-helical spikes extend away from said platform.

4. The apparatus of claim 1 wherein at least a portion of each of said at least two helical spikes is made of a shape memory alloy that is responsive to changes in temperature above and below a predetermined temperature transition range, said at least two helical spikes being heated above said predetermined temperature transition range as said at least two helical spikes are being implanted into bone.

5. The apparatus of claim 4 further comprising a tubular sleeve for receiving said anchor, said anchor being positionable inside said tubular sleeve when the temperature of said at least two helical spikes is below said predetermined transition temperature range.

6. The apparatus of claim 1 further comprising a cannula through which said anchor is insertable and for accessing one of the first and second bones.

7. The apparatus of claim 1 wherein each of said at least two helical spikes has a connecting portion at a proximal end connected to said platform and an intermediate portion extending between said connecting portion and said tip portion.

8. The apparatus of claim 7 comprising a pair of helical spikes extending around said longitudinal axis, said proximal ends of said pair of helical spikes being spaced 180° apart.

9. The apparatus of claim 7 comprising three helical spikes extending around said longitudinal axis, said proximal ends of said three helical spikes being spaced 120° apart.

10. The apparatus of claim 1 wherein said first surface has a shape that is complimentary to the shape of an outer surface of the bone for engaging the outer surface of the bone.

11. The apparatus of claim 1 wherein one of said first and second bones is a sacrum and the other of said first and second bones is a fifth lumbar (L5) vertebrae.

12. The apparatus of claim 11 wherein said first surface is porous to promote bone in-growth.

13. The apparatus of claim 11 wherein said first surface has surface features that elevate its surface area to promote bone in-growth.

14. The apparatus of claim 11 wherein, when said anchor is in said second condition, at least a portion of said platform is recessed into an outer surface of one of the sacrum or the L5 vertebrae.

15. The apparatus of claim 11 wherein said first surface has an oblique shape that is complimentary to the shape of an outer surface of one of the sacrum or the L5 vertebrae.

16. The apparatus of claim 1 wherein said first surface has a rough texture that provides an increased surface area to promote bone in-growth.

17. The apparatus of claim 1 wherein each of said at least two helical spikes has a solid cross-section.

18. The apparatus of claim 1 wherein each of said at least two helical spikes has a tubular cross-section.

19. The apparatus of claim 1 wherein a first section of each of said at least two helical spikes has a solid cross-section and a second section of each of said at least two helical spikes has a tubular cross-section.

20. The apparatus of claim 1 further comprising a starter tool for forming starting holes in the bone that said at least two helical spikes are received in, said starter tool comprising a platform having a surface that extends transverse to a longitudinal axis of said starter tool and at least two helical spikes extending from said surface, said at least two helical spikes on said starter tool corresponding in quantity and size to said at least two helical spikes on said anchor but are substantially shorter in axial length to resist radially outward deformation during rotation of said platform.

21. An apparatus for attaching a fifth lumbar (L5) vertebrae to a sacrum, said apparatus comprising:
an anchor for extending between the L5 vertebrae and the sacrum and for attaching the L5 vertebrae to the sacrum, said anchor having a platform for drivingly rotating said anchor, said platform including a first surface that is solid and that extends generally transverse to a longitudinal axis of said anchor;
said anchor further having at least two helical spikes for embedding into both of the L5 vertebrae and the sacrum upon rotation of said platform, said at least two helical spikes projecting from said first surface of said platform and extending around said longitudinal axis, said at least two helical spikes having a tip portion at a distal end for penetrating into at least one of the L5 vertebrae and the sacrum as said platform is rotated;
said anchor having a first condition in which said at least two helical spikes are embeddable into one of the L5 vertebrae and the sacrum, said anchor further having a second condition in which said at least two helical spikes are embeddable into both of the L5 vertebrae and the sacrum to attach the L5 vertebrae and the sacrum to one another while maintaining an intervertebral space between the L5 vertebrae and the sacrum, said anchor being movable from said first condition to said second condition by rotation of said platform;
a portion of each of said at least two helical spikes of said anchor, when said anchor is embedded into the L5 vertebrae and the sacrum, extending across the intervertebral space between the L5 vertebrae and the sacrum.

22. The apparatus of claim 21 wherein said first surface is porous to promote bone in-growth.

23. The apparatus of claim 21 wherein said first surface has surface features that elevate its surface area to promote bone in-growth.

24. The apparatus of claim 21 wherein, when said anchor is in said second condition, at least a portion of said platform is recessed into an anterior surface of the sacrum.

25. The apparatus of claim 21 wherein said first surface has an oblique shape that is complimentary to the shape of an anterior surface of the sacrum for engaging the anterior surface.

26. The apparatus of claim 21 wherein, when said anchor is in said second condition, at least a portion of said platform is recessed into a posterior surface of the sacrum.

27. The apparatus of claim 21 wherein said first surface has an oblique shape that is complimentary to the shape of a posterior surface of the sacrum for engaging the posterior surface.

28. The apparatus of claim 21 wherein, when said anchor is in said second condition, at least a portion of said platform is recessed into an anterior surface of the L5 vertebrae.

29. The apparatus of claim 21 wherein said first surface has an oblique shape that is complimentary to the shape of an anterior surface of the L5 vertebrae for engaging the anterior surface.

30. The apparatus of claim 21 wherein each of said at least two helical spikes has a cylindrical shape with a generally constant overall diameter.

31. The apparatus of claim 21 wherein each of said at least two helical spikes, when implanted, has a conical shape that increases in diameter as said at least two helical spikes extend away from said platform.

32. The apparatus of claim 31 wherein at least a portion of each of said at least two helical spikes is made of a shape memory alloy that is responsive to changes in temperature above and below a predetermined temperature transition range, said at least two helical spikes being heated above said predetermined temperature transition range as said at least two helical spikes are being implanted into the bone.

33. The apparatus of claim 21 wherein each of said at least two helical spikes has a connecting portion at a proximal end connected to said platform and an intermediate portion extending between said connecting portion and said tip portion.

34. The apparatus of claim 33 comprising a pair of helical spikes extending around said longitudinal axis, said proximal ends of said pair of helical spikes being spaced 180° apart.

35. The apparatus of claim 33 comprising three helical spikes extending around said longitudinal axis, said proximal ends of said three helical spikes being spaced 120° apart.

36. The apparatus of claim 21 wherein each of said at least two helical spikes has a solid cross-section.

37. The apparatus of claim 21 wherein each of said at least two helical spikes has a tubular cross-section.

38. The apparatus of claim 21 wherein a first portion of each of said at least two helical spikes has a solid cross-section and a second section of each of said at least two helical spikes has a tubular cross-section.

39. A method for attaching a first bone in a patient's body to an adjacent second bone, the second bone being separated from the first bone by a space between the bones, said method comprising the steps of:
providing an anchor having a platform and at least two helical spikes, the platform having a first surface that extends generally transverse to a longitudinal axis of the anchor, the at least two helical spikes projecting from the first surface of the platform and extending around the longitudinal axis;
engaging one of the bones with the at least two helical spikes;
rotating the platform of the anchor which embeds a first portion of each of the at least two helical spikes into one of the first and second bones;
further rotating the platform of the anchor so that the anchor extends across the space and embeds the first portion of the anchor into the other of the first and second bones and a second portion of the at least two helical spikes to attach the first and second bones to one another while maintaining the space between the bones, a portion of each of the at least two helical spikes extending across the space between the bones.

40. The method of claim 39 wherein said step of rotating the platform embeds the at least two helical spikes into one of the first and second bones and said step of further rotating the platform embeds the at least two helical spikes into both of the first and second bones.

41. The method of claim 39 wherein at least a portion of each of the at least two helical spikes is made of a shape memory alloy that is responsive to changes in temperature above and below a predetermined temperature transition range, said method further comprising the step of heating the at least two helical spikes above the predetermined temperature transition range as the at least two helical spikes are being implanted into bone.

42. The method of claim 39 wherein one of said first and second bones is a sacrum and the other of said first and second bones is a fifth lumbar (L5) vertebrae, further comprising the step of rotating the platform until the first surface of the anchor engages a surface on one of the sacrum and the L5 vertebrae.

43. The method of claim 42 further comprising the step of rotating the platform until at least a portion of the platform is recessed into the surface on one of the sacrum and the L5 vertebrae.

44. The method of claim 39 further including the step of forming at least two holes in one of the bones, said step of engaging the bone with the at least two helical spikes includes moving an end portion of each of said at least two helical spikes into one of the at least two holes in the bone.

45. The method of claim 44 further including the step of providing a starter tool having at least two helical spikes that correspond in quantity and size to the at least two helical spikes on said anchor, the at least two helical spikes on the starter tool having a short axial length to resist radially outward deformation, said step of forming at least two holes includes rotating the starter tool to form the holes.

46. A method as set forth in claim 45 further comprising the steps of:
positioning a wire through the first and second bones along a desired axis;
placing the starter tool over the wire and sliding the starter tool toward one of the bones along the desired axis;
engaging a surface of one bone with the at least two helical spikes on the starter tool and rotating the starter tool to form the at least two holes;
removing the starter tool from the wire;
placing the anchor over the wire and sliding the anchor toward the one bone along the desired axis; and
inserting the at least two helical spikes on the anchor into the at least two holes in the one bone formed by the starter tool.

47. The method of claim 45 further comprising the steps of:
positioning a wire through the first and second bones along a desired axis; and
placing the anchor over the wire and sliding the anchor toward one of the bones along the desired axis.

48. The method of claim 39 further including the step of limiting radially outward deformation of the at least two helical spikes by positioning a sleeve around the helical spikes during rotation of the anchor about a central axis of the anchor.

49. A method for attaching a fifth lumbar (L5) vertebrae to a sacrum, said method comprising the steps of:
removing disc material disposed between the L5 vertebrae and the sacrum to create an interbody space;
providing an anchor for extending between the L5 vertebrae and the sacrum and for attaching the L5 vertebrae to the sacrum, the anchor having a platform for drivingly rotating the anchor, the platform including a first surface that extends generally transverse to a longitudinal axis of the anchor;
the anchor further having at least two helical spikes for embedding into both of the L5 vertebrae and the sacrum upon rotation of the platform, the at least two helical spikes projecting from the first surface and extending around the longitudinal axis;
engaging one of the L5 vertebrae and the sacrum with the at least two helical spikes on the anchor;
rotating the platform so that a portion of each of the at least two helical spikes embeds into one of the sacrum and the L5 vertebrae;
further rotating the platform so that the at least two helical spikes extend across the interbody space and into the other of the sacrum and the L5 vertebrae to attach the L5 vertebrae and the sacrum to each other while maintaining the interbody space between the L5 vertebrae and the sacrum such that a portion of each of the at least two helical spikes extends across the interbody space between the L5 vertebrae.

50. The method of claim 49 further comprising the step of rotating the platform until at least a portion of the platform is recessed into a surface on one of the sacrum and the L5 vertebrae.

51. The method of claim 50 further comprising the step of inserting the anchor into the patient's body through a cannula.

52. The method of claim 49 further comprising the step of placing an osteogenic material into the interbody space following implantation of the anchor.
